(19) United States
(12) Patent Application Publication
(10) Pub. No.: US 2025/0242140 A1
(43) Pub. Date: Jul. 31, 2025

(54) Skin Preparation Device

(71) Applicant: Becton, Dickinson and Company, Franklin Lakes, NJ (US)

(72) Inventors: Shishir Prasad, Ramsey, NJ (US); Manish Kumar, Bengaluru (IN); Karthik MR, Bangalore (IN)

(21) Appl. No.: 18/853,958

(22) PCT Filed: Mar. 31, 2023
(86) PCT No.: PCT/US23/17073

§ 371 Date: Oct. 3, 2024

Related U.S. Application Data
(60) Provisional application No. 63/327,199, filed on Apr. 4, 2022.

(51) Int. Cl. A61M 35/00

(52) U.S. Cl. CPC A61M 35/006; A61M 2210/04

(57) ABSTRACT

A skin preparation device for applying an antiseptic composition to a skin surface of a patient that may include an applicator configured to absorb the antiseptic composition, the applicator comprising an application surface and a holding surface, the application surface configured to apply the antiseptic composition to the skin surface of the patient; and a holding member configured to provide a portion of the skin preparation device that is gripped by a user when using the skin preparation device, the holding member including a first end and an opposing second end, the holding member being operatively connected to the holding surface of the applicator, wherein the holding member is made of a material that provides flexibility to allow the first end and the second end to be pulled into contact with one another to be gripped by a user of the skin preparation device.

### CROSS-REFERENCE TO RELATED APPLICATION

The present application claims priority to U.S. Provisional Application Ser. No. 63/327,199, entitled “Skin Preparation Device”, filed Apr. 4, 2022, the entire disclosure of which is hereby incorporated by reference in its' entirety.

### BACKGROUND OF THE INVENTION

### Field of the Invention

The present disclosure is directed to a skin disinfectant wipe and, more particularly, is directed to a skin disinfectant wipe that reduces or eliminates contact between a user's skin surface and a patient's skin surface when applying the disinfectant to the patient's skin surface.

### Description of Related Art

Proper cleansing and disinfecting of a patient's skin, for example but not limited to, when preparing for injection or aspiration through skin or a surgery site is crucial to reduce the likelihood of adverse events, including infection, during and following the intervention. However, for many reasons ranging from pressure to rapidly prepare a patient for these procedures to distractions inherent in the environment, many clinicians and other staff do not engage in proper cleansing and disinfecting techniques, for example such as the suggested guidelines of 30 seconds of cleansing, followed by 30 seconds of drying for dry skin areas.

A current method for patient skin preparation is dominated by the use of disinfectant wipes. These disinfectant wipes tend to lead to accidental touching of the patient's skin by the user applying the disinfectant wipe and improper aseptic technique, which both lead to contamination and increased rate of infection on the patient's skin surface.

### SUMMARY OF THE INVENTION

In view of the foregoing disclosure, there is a current need in the art for a disinfectant wipe that reduces or eliminates contact between a user's skin surface and a patient's skin surface when applying the disinfectant to the patient's skin surface. A further need exists in the art for a disinfectant wipe that reduces or eliminates contact between a user's skin surface and a patient's skin surface when applying the disinfectant to the patient's skin surface and remains cost-effective for mass production.

In some non-limiting embodiments and aspects of the present disclosure, a skin preparation device for applying an antiseptic composition to a skin surface of a patient may include an applicator configured to absorb the antiseptic composition, the applicator comprising an application surface and a holding surface, the application surface configured to apply the antiseptic composition to the skin surface of the patient; and a holding member configured to provide a portion of the skin preparation device that is gripped by a user when using the skin preparation device, the holding member including a first end and an opposing second end, the holding member being operatively connected to the holding surface of the applicator, wherein the holding member is made of a material that provides flexibility to allow the first end and the second end to be pulled into contact with one another to be gripped by the user of the skin preparation device.

In some non-limiting embodiments or aspects of the present disclosure, the applicator and the holding member may be made of different materials. The applicator may be made of foam or non-woven paper. The holding member may be made of a blister packaging. The applicator may have a thickness that prevents the holding member from contacting the skin surface of the patient. The applicator may include a flexibility to bend as the first end and the second end of the holding member are pulled into contact with one another. The holding surface of the applicator may define a plurality of grooves and ridges to assist the applicator in bending. The applicator may be operatively connected to the holding member using an adhesive. The applicator may be rectangular in shape. The antiseptic composition may include alcohol based compounds, non-alcohol based compounds, or a mixture of alcohol and non-alcohol based compounds.

In some non-limiting embodiments and aspects of the present disclosure, a skin preparation device for applying an antiseptic composition to a skin surface of a patient may include an applicator configured to absorb the antiseptic composition, the applicator comprising an application surface and a holding surface, the application surface configured to apply the antiseptic composition to the skin surface of the patient; a holding member configured to provide a portion of the skin preparation device that is gripped by a user when using the skin preparation device, the holding member including a first end and an opposing second end, the holding member being operatively connected to the holding surface of the applicator; and a packaging web operatively connected to the holding member to seal the applicator between the packaging web and the holding member.

In some non-limiting embodiments or aspects of the present disclosure, the holding member may be made of a material that provides flexibility to allow the first end and the second end to be pulled into contact with one another to be gripped by the user of the skin preparation device. The packaging web may include a peel tab to remove the packaging web from the holding member. The packaging web may include at least one perforation line to assist in removing at least a portion of the packaging web from the skin preparation device. The packaging web may be heat sealed to a perimeter of the holding member. The packaging web may be made of blister packaging. The applicator may be made of foam or non-woven paper. The applicator may have a thickness that prevents the holding member from contacting the skin surface of the patient. The applicator may include a flexibility to bend as the first end and the second end of the holding member are pulled into contact with one another. The holding surface of the applicator may define a plurality of grooves and ridges to assist the applicator in bending.

The present invention is also described in relation to the following clauses:

Clause 1: A skin preparation device for applying an antiseptic composition to a skin surface of a patient, the skin preparation device comprising: an applicator configured to absorb the antiseptic composition, the applicator comprising an application surface and a holding surface, the application surface configured to apply the antiseptic composition to the skin surface of the patient; and a holding member configured to provide a portion of the skin preparation device that is gripped by a user when using the skin preparation device, the holding member including a first end and an opposing second end, the holding member being operatively connected to the holding surface of the applicator, wherein the holding member is made of a material that provides flexibility to allow the first end and the second end to be pulled into contact with one another to be gripped by the user of the skin preparation device.

Clause 2: The skin preparation device of Clause 1, wherein the applicator and the holding member are made of different materials.

Clause 3: The skin preparation device of Clause 1 or 2, wherein the applicator is made of foam or non-woven paper.

Clause 4: The skin preparation device of any of Clauses 1-3, wherein the holding member is made of a blister packaging.

Clause 5: The skin preparation device of any of Clauses 1-4, wherein the applicator has a thickness that prevents the holding member from contacting the skin surface of the patient.

Clause 6: The skin preparation device of any of Clauses 1-5, wherein the applicator includes a flexibility to bend as the first end and the second end of the holding member are pulled into contact with one another.

Clause 7: The skin preparation device of Clause 6, wherein the holding surface of the applicator defines a plurality of grooves and ridges to assist the applicator in bending.

Clause 8: The skin preparation device of any of Clauses 1-7, wherein the applicator is operatively connected to the holding member using an adhesive.

Clause 9: The skin preparation device of any of Clauses 1-8, wherein the applicator is rectangular in shape.

Clause 10: The skin preparation device of any of Clauses 1-9, wherein the antiseptic composition comprises alcohol based compounds, non-alcohol based compounds, or a mixture of alcohol and non-alcohol based compounds.

Clause 11: A skin preparation device for applying an antiseptic composition to a skin surface of a patient, the skin preparation device comprising: an applicator configured to absorb the antiseptic composition, the applicator comprising an application surface and a holding surface, the application surface configured to apply the antiseptic composition to the skin surface of the patient; a holding member configured to provide a portion of the skin preparation device that is gripped by a user when using the skin preparation device, the holding member including a first end and an opposing second end, the holding member being operatively connected to the holding surface of the applicator; and a packaging web operatively connected to the holding member to seal the applicator between the packaging web and the holding member.

Clause 12: The skin preparation device of Clause 11, wherein the holding member is made of a material that provides flexibility to allow the first end and the second end to be pulled into contact with one another to be gripped by the user of the skin preparation device.

Clause 13: The skin preparation device of Clause 11 or 12, wherein the packaging web comprises a peel tab to remove the packaging web from the holding member.

Clause 14: The skin preparation device of any of Clauses 11-13, wherein the packaging web comprises at least one perforation line to assist in removing at least a portion of the packaging web from the skin preparation device.

Clause 15: The skin preparation device of any of Clauses 11-14, wherein the packaging web is heat sealed to a perimeter of the holding member.

Clause 16: The skin preparation device of any of Clauses 11-15, wherein the packaging web is made of blister packaging.

Clause 17: The skin preparation device of any of Clauses 11-16, wherein the applicator is made of foam or non-woven paper.

Clause 18. The skin preparation device of any of Clauses 11-17, wherein the applicator has a thickness that prevents the holding member from contacting the skin surface of the patient.

Clause 19: The skin preparation device of any of Clauses 11-18, wherein the applicator includes a flexibility to bend as the first end and the second end of the holding member are pulled into contact with one another.

Clause 20: The skin preparation device of Clause 19, wherein the holding surface of the applicator defines a plurality of grooves and ridges to assist the applicator in bending.

### BRIEF DESCRIPTION OF THE DRAWINGS

FIG. 1 is a perspective view of a skin preparation device according to a non-limiting embodiment or aspect of the present disclosure,

FIG. 2 is an exploded view of the skin preparation device of FIG. 1.

FIG. 3 is a perspective view of an applicator and holding member of the skin preparation device of FIG. 1.

FIG. 4 is a perspective view of the applicator and holding member of FIG. 3 in an application position,

FIG. 5 is a perspective view of a skin preparation device according to one non-limiting embodiment or aspect of the present disclosure,

FIG. 6 is a schematic view of a method of using a skin preparation device according to a non-limiting embodiment or aspect of the present disclosure, and

FIG. 7 is a schematic view of a method of assembling a skin preparation device according to a non-limiting embodiment or aspect of the present disclosure.

### DESCRIPTION OF THE DISCLOSURE

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates, and grammatical variants of those words or phrases.

The figures accompanying this application are representative in nature, and should not be construed as implying any particular scale or directionality, unless otherwise indicated. For purposes of the description hereinafter, the terms “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. Hence, specific dimensions and other physical characteristics related to the embodiments disclosed herein are not to be considered as limiting.

Provided herein is a device for applying an antiseptic composition to skin of a patient, for example, for preparing an area of the patient's skin for injection or aspiration through skin, or a surgical intervention. As used herein, the term “patient” is any animal, including humans, and a “human patient” is any human. As used herein, the term “antiseptic composition” is any composition that prevents or inhibits the growth of one or more microorganisms on the patient's skin surface.

As used herein, the term “surgical intervention” means any percutaneous treatment (e.g., catheterization, angioplasty, needle biopsy, and the like), open surgery, laparoscopic surgery, and/or minimally-invasive surgery that involves puncturing the skin or creating one or more incisions of varying size in the skin of the patient.

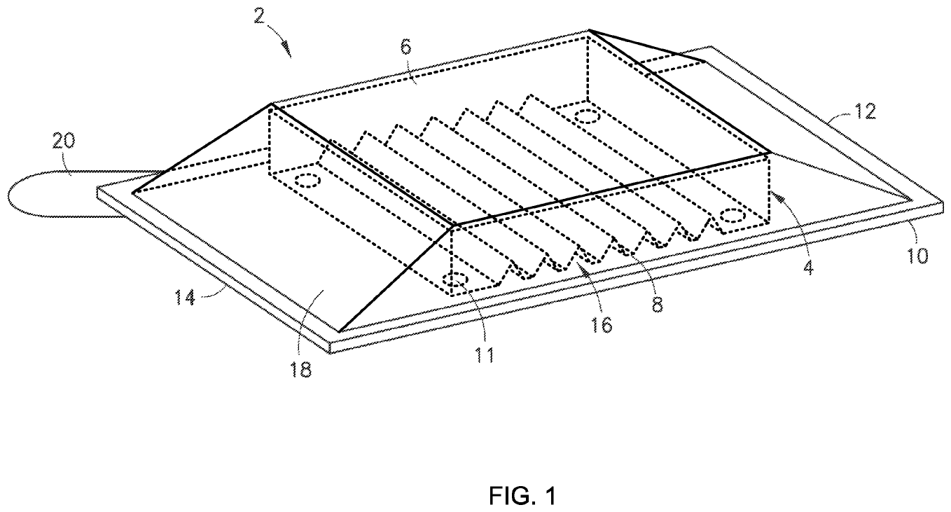

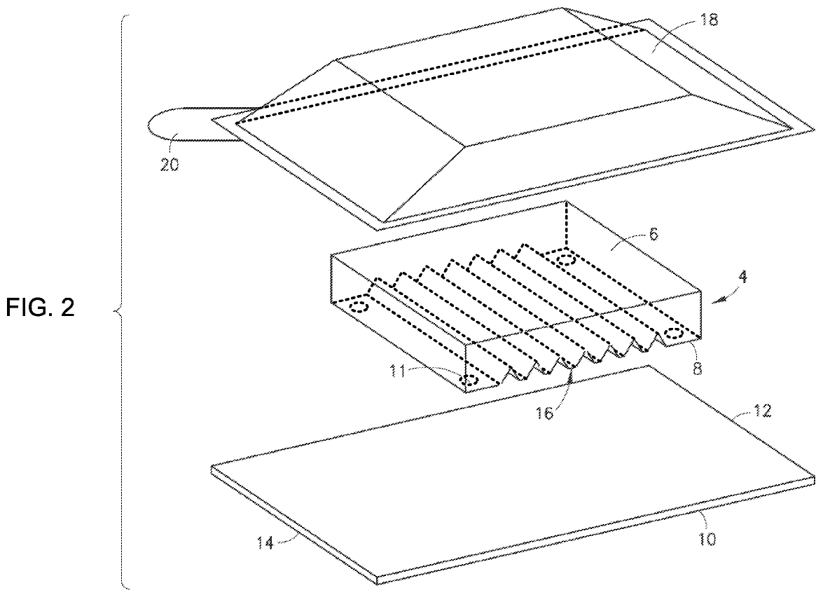

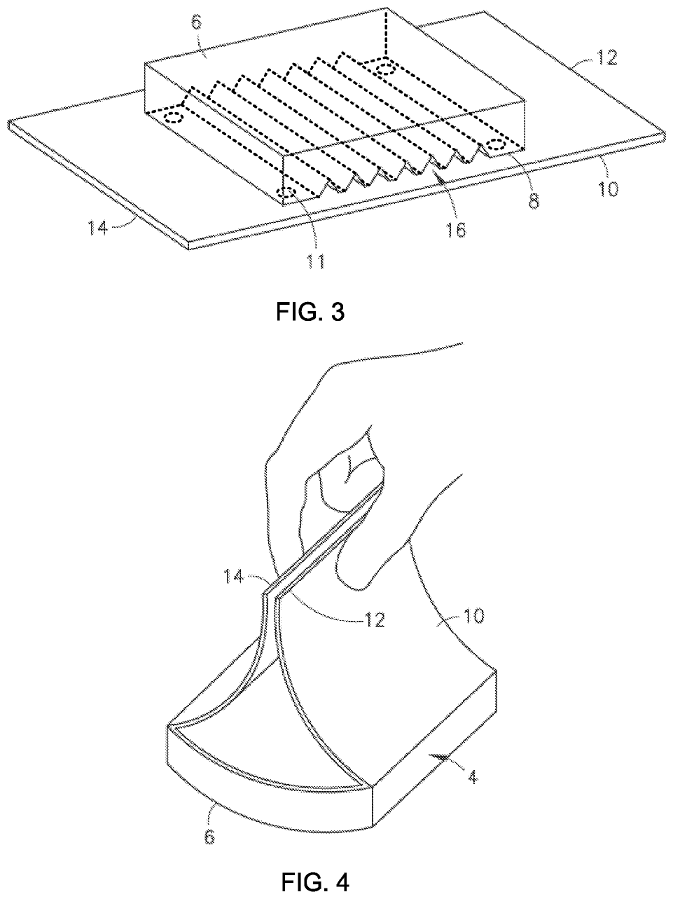

In some non-limiting embodiments or aspects, as shown in FIGS. 1-4, a skin preparation device 2 according to the present disclosure is shown and described in detail. The skin preparation device 2 may include an applicator 4, such as a sponge, foam, or other porous, absorbent material that is configured to hold an antiseptic composition, and allows a user to apply the antiseptic composition to the patient's skin at the site of the surgical intervention and, optionally, one or more areas adjacent to the site of the surgical intervention. In one non-limiting embodiment or aspect, the applicator 4 may be made of a foam, such as polyurethane or polystyrene, or a non-woven paper. The applicator 4 can be a natural sponge, a synthetic sponge including, for example and without limitation, a polyurethane, a polyester, and/or a vegetal cellulose, or other suitable material, so long as the material is capable of absorbing and/or dispensing the antiseptic composition. The applicator 4 may have an application surface 6 and an opposing holding surface 8. In one example, the applicator 4 may be a disinfectant wipe. In one non-limiting embodiment or aspect, the applicator 4 may be substantially rectangular in shape. In other examples, the applicator 4 may be oval or circular in shape. The applicator 4 may be presoaked with the antiseptic composition.

In non-limiting embodiments or aspects, the antiseptic composition includes one or more alcohols, such as ethyl alcohol, propyl alcohol, isopropyl alcohol, n-propanol, and/or mixtures thereof. In non-limiting embodiments or aspects, the antiseptic composition includes one or more non-alcohol based compounds, such as iodine, para-chloro-meta-xylenol, bis-biguanides such as chlorhexidine gluconate (CHG), chlorhexidine diacetate or quaternium class compounds such as benzethonium chloride, benzalkonium chloride, chloroxylenol, triclosan, hexachlorophenes, octenidine, diazolidinyl urea, methyl chloro isothiazoline, methyl isothiazoline, triclosan, and/or mixtures thereof. In non-limiting embodiments or aspects, the antiseptic composition includes a mixture of any of the aforementioned, including mixtures of alcohol and non-alcohol based compounds. In non-limiting embodiments or aspects, the antiseptic composition includes CHG and an alcohol, for example isopropyl alcohol. In non-limiting embodiments or aspects, the antiseptic composition includes about 2% (w/v) CHG and about 70% (v/v) isopropyl alcohol.

In non-limiting embodiments or aspects, the antiseptic composition is effective against one or more microorganisms, such as bacteria, viruses, and/or fungi. In non-limiting embodiments or aspects, the microorganism is one or more of coagulase-negative staphylococci, Staphylococcus aureus (including methicillin-resistant S. aureus), Enterococcus spp. (including vancomycin-resistant Enterococci, such as E. faecium), Candida spp., Escherichia coli (including extended-spectrum cephalosporin resistant E. coli and carbapenem-resistant E. coli), Clostridium difficile, Pseudomonas aeruginosa (including carbapenem-resistant P. aeruginosa), Klebsiella pneumoniae (including extended-spectrum cephalosporin-resistant K. pneumoniae and carbapenem-resistant K. pneumoniae), Enterobacter spp., Acinetobacter spp. (including Acinetobacter baumannii), and Klebsiella oxytoca.

In non-limiting embodiments or aspects, the skin preparation device 2 may include a holding member 10 to allow a user to apply the antiseptic composition to the skin of a patient, while maintaining aseptic technique (e.g., by not directly contacting the applicator or the patient's skin surface). In one example, the holding member 10 may be a substantially flexible planar member that is operatively connected to the holding surface 8 of the applicator 4. The holding member 10 may be adhesively attached to or integrally formed with the holding surface 8 of the applicator 4 at connection points 11. In one non-limiting embodiment or aspect, the connection points 11 may be positioned at each corner of the holding surface 8 of the applicator 4. However, it is to be understood that the connection points 11 may be positioned at any location on the holding surface 8 of the applicator 4. The connection points 11 may be locations in which an adhesive is applied to the holding surface 8 to operatively connect the applicator 4 to the holding member 10.

The holding member 10 may be made from a material that permits a user to bend the ends 12, 14 of the holding member 10 towards one another to create a gripping handle for the user to grip to use the skin preparation device 2. As shown in FIG. 4 in one non-limiting embodiment or aspect, the ends 12, 14 may be bent or rotated in a direction away from the holding surface 8 or, more generally, the applicator 4. The ends 12, 14 of the holding member 10 may be pulled into contact with one another so the user can squeeze the ends 12, 14 between two of his/her fingers. In this holding configuration, the ends 12, 14 of the holding member 10 may be gripped by the user, while an intermediate portion of the holding member 10 remains operatively connected to the applicator 4.

As shown in FIGS. 1-4, according to some non-limiting embodiments or aspects of the present disclosure, the application surface 6 of the applicator 4 may be substantially planar and smooth to apply the antiseptic composition to the patient's skin surface. The holding surface 8 of the applicator 4 may include a plurality of grooves and ridges 16. In one non-limiting embodiment or aspect of the present disclosure, the plurality of grooves and ridges 16 may be provided on the applicator 4 to assist in the folding of the holding member 10. The plurality of grooves and ridges 16 permit the applicator 4 to bend as the ends 12, 14 are pulled towards one another to be gripped by the user. The plurality of grooves and ridges 16 provide greater flexibility to the applicator 4 to permit the applicator 4 to bend in on itself as the ends 12, 14 are rotated into the holding position for the user. Due to the relative rigidity of the holding member 10 and the relative flexibility of the applicator 4, the applicator 4 can be bent to squeeze the antiseptic composition from the applicator 4 while not rupturing the applicator 4. The foam material stretchability of the applicator 4 may be optimized by adjusting the foam cross-linking density. Foam cross-linking density and size also may influence the amount of antiseptic composition the applicator 4 can hold.

By using the holding member 10 to grip the skin preparation device 2, the user avoids making contact with the patient's skin surface and/or the applicator 4 and, thereby, avoiding contaminating the patient's skin or the applicator 4. Furthermore, as the ends 12, 14 of the holding member 10 are pulled into contact with one another to be gripped by the user, the applicator 4 is bent with the holding member 10, which results in the antiseptic composition being squeezed out of the applicator 4 to assist in effectively coating the patient's skin surface with the antiseptic composition. Furthermore, a thickness of the applicator 4 may be sufficiently large enough to create a distance between the patient's skin surface and the non-sterile region of the holding member 10. The thickness of the applicator 4 may range from several centimeters, such as two centimeters, to several microns, such as three microns. The use of the skin preparation device 2 also complies to the Aseptic Non Touch Technique (ANTT) due to only the holding member 10 being gripped by the user and not the applicator 4 that includes the antiseptic composition. No additional components are needed for the skin preparation device 2 as compared to current disinfectant wipes, resulting in a low cost solution to the listed deficiencies with current disinfectant wipes.

With reference to FIGS. 1-4, according to some non-limiting embodiments or aspects, the skin preparation device 2 may further include a packaging web 18 operatively connected to the holding member 10 to create a sterile packaging for the applicator 4. In one non-limiting embodiment or aspect, the holding member 10 and the packaging web 18 may be made of the same material, such as a flexible sterile medical grade plastic. The packaging web 18 may be heat sealed to the holding member 10 around the entire perimeter of the packaging web 18 to ensure the inner cavity of the skin preparation device 2 that holds the applicator 4 is kept sterile from any contaminates outside the skin preparation device 2. The packaging web 18 may be include a peel tab 20 to assist in removing the packaging web 18 from the holding member 10. In one non-limiting embodiment or aspect, the peel tab 20 may be provided on a corner of the packaging web 18. As the peel tab 20 is pulled away from the holding member 10, the heat seal between the holding member 10 and the packaging web 18 is broken and the packaging web 18 is removed from the holding member 10. In one non-limiting embodiment or aspect, the holding member 10 and the packaging web 18 may form a blister packaging for the skin preparation device 2. The holding member 10 and the packaging web 18 may be made of a material that has negligible disinfectant permeability, such as a metal coated packaging film material. The packaging web 18 may include written or pictorial directions to a user to open the packaging web 18 from an edge or a corner, such as the peel tab 20, to pull the packaging web 18 away from the holding member 10.

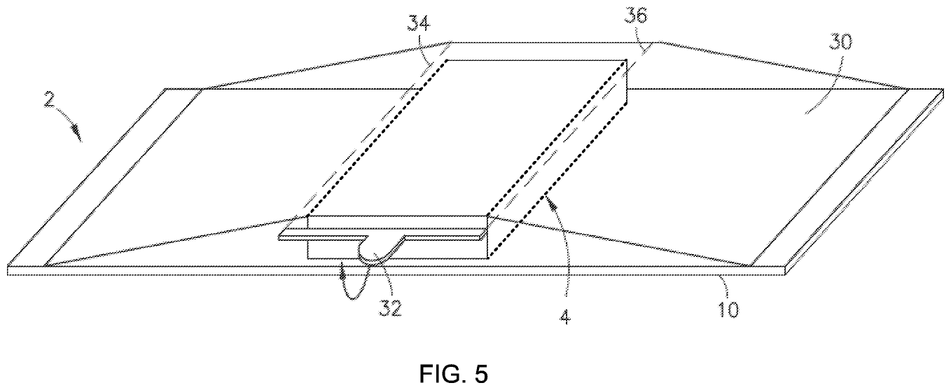

With reference to FIG. 5, according to another non-limiting embodiment or aspect of the present disclosure, a packaging web 30 may be heat sealed to the holding member 10 around a perimeter of the packaging web 30. In this example of the packaging web 30, a peel tab 32 is provided on an intermediate portion of the packaging web 30 that covers the applicator 4. A pair of opposing perforation lines 34, 36 are provided on this intermediate portion of the packaging web 30 to assist in removing the intermediate portion of the packaging web 30 when the peel tab 32 is pulled away from the packaging web 30. As the peel tab 32 is pulled away, the perforation lines 34, 36 are broken, permitting the intermediate portion of the packaging web 30 to be removed from the skin preparation device 2. With the intermediate portion of the packaging web 30 removed from the skin preparation device 2, the application surface 6 of the applicator 4 is exposed to apply the antiseptic composition to the patient's skin surface. By keeping portions of the packaging web 30 operatively connected to the holding member 10, the remaining portions of the packaging web 30 may assist in keeping the applicator 4 sterile and free of contaminants outside of the skin preparation device 2.

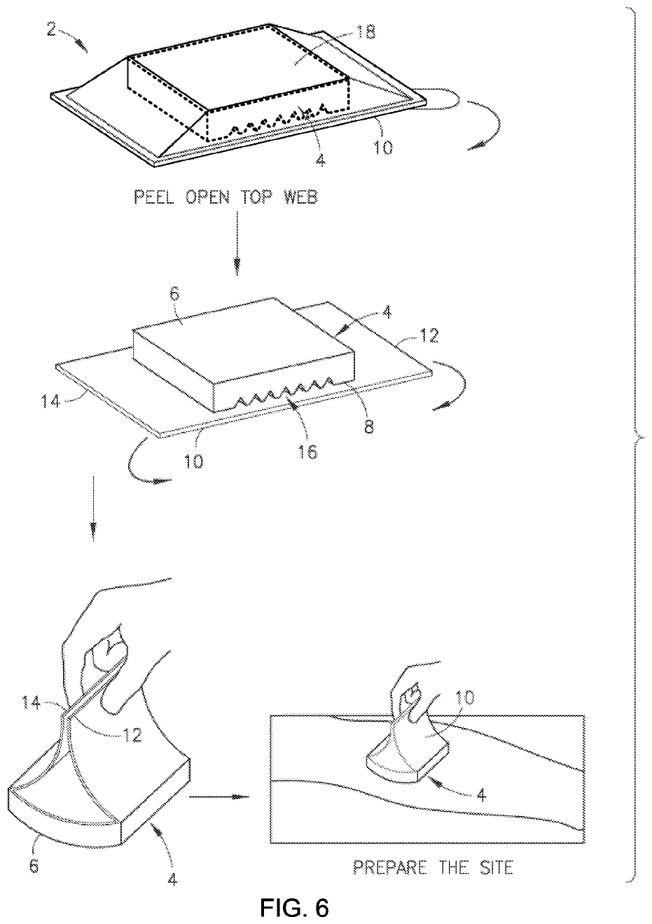

With reference to FIG. 6, according to one non-limiting embodiment or aspect of the present disclosure, a method of preparing and using the skin preparation device 2 is shown and described. According to this method, the peel tab 20 is pulled by a user to remove the packaging web 18 from the holding member 10. After the packaging web 18 has been removed from the holding member 10, a user may grip the ends 12, 14 of the holding member 10 and pull the ends 12, 14 of the holding member 10 towards one another. The ends 12, 14 are then folded in contact together to be gripped between at least two fingers of the user. As the user grips the ends 12, 14 together, the applicator 4 may be pressed against the patient's skin surface to apply the antiseptic composition to the patient's skin surface.

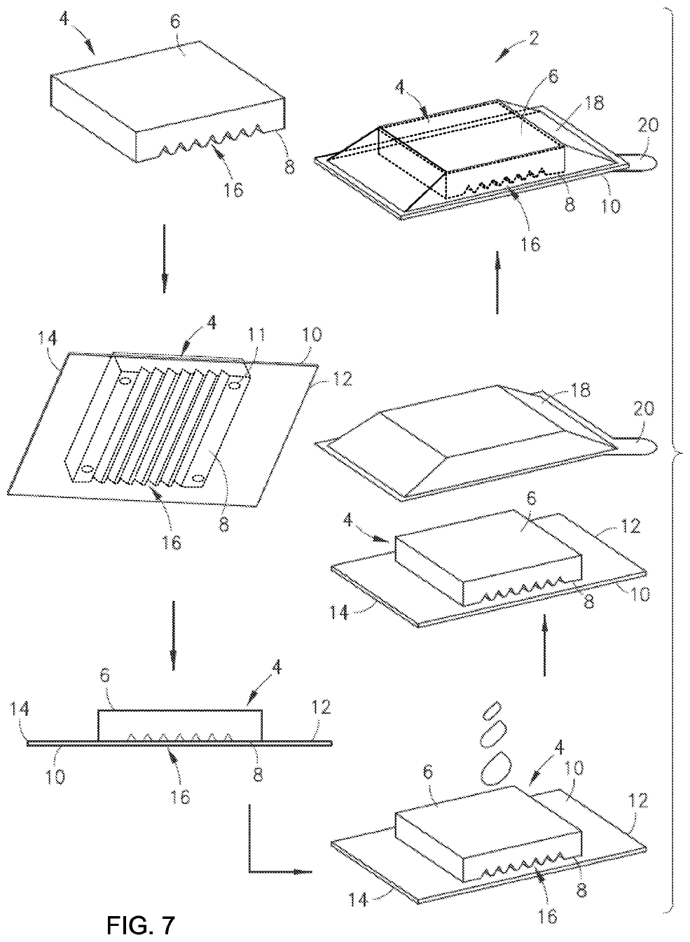

With reference to FIG. 7, according to one non-limiting embodiment or aspect of the present disclosure, a method of manufacturing the skin preparation device 2 is shown and described. The applicator 4 is cut from a sponge, foam, or similar absorbent material, such as a non-woven paper. An adhesive is then applied to the connection points 11 on the applicator 4. The applicator 4 is then pressed against the holding member 10 and the adhesive is cured to operatively connect the applicator 4 to the holding member 10. Once the applicator 4 is connected to the holding member 10, the antiseptic composition may be applied to the applicator 4 to allow the antiseptic composition to soak into the applicator 4. Once the antiseptic composition has been applied to the applicator 4, the packaging web 18 may be placed over the applicator 4 and brought into contact with the holding member 10. The packaging web 18 may then be heat sealed to the perimeter of the holding member 10 to seal the skin preparation device 2. It is also to be understood that some of these steps may be performed in a different order, including applying the antiseptic composition to the applicator 4 before operatively connecting the applicator 4 to the holding member 10.

The present disclosure provides an improved combination of device design, material properties, and packaging features to address the shortcomings of wipes in skin preparation, i.e. being able to aseptic non-touch application of skin preparation device.

While the present disclosure has been described in terms of the above detailed description, those of ordinary skill in the art will understand that alterations may be made within the spirit of the invention. Accordingly, the above should not be considered limiting, and the scope of the invention is defined by the appended claims.

The invention claimed is:

1. A skin preparation device for applying an antiseptic composition to a skin surface of a patient, the skin preparation device comprising:
an applicator configured to absorb the antiseptic composition, the applicator comprising an application surface and a holding surface, the application surface configured to apply the antiseptic composition to the skin surface of the patient; and
a holding member configured to provide a portion of the skin preparation device that is gripped by a user when using the skin preparation device, the holding member including a first end and an opposing second end, the holding member being operatively connected to the holding surface of the applicator, wherein the holding member is made of a material that provides flexibility to allow the first end and the second end to be pulled into contact with one another to be gripped by the user of the skin preparation device.

2. The skin preparation device of claim 1, wherein the applicator and the holding member are made of different materials.

3. The skin preparation device of claim 1, wherein the applicator is made of foam or non-woven paper.

4. The skin preparation device of claim 1, wherein the holding member is made of a blister packaging.

5. The skin preparation device of claim 1, wherein the applicator has a thickness that prevents the holding member from contacting the skin surface of the patient.

6. The skin preparation device of claim 1, wherein the applicator includes a flexibility to bend as the first end and the second end of the holding member are pulled into contact with one another.

7. The skin preparation device of claim 6, wherein the holding surface of the applicator defines a plurality of grooves and ridges to assist the applicator in bending.

8. The skin preparation device of claim 1, wherein the applicator is operatively connected to the holding member using an adhesive.

9. The skin preparation device of claim 1, wherein the applicator is rectangular, oval, or circular in shape.

10. The skin preparation device of claim 1, wherein the antiseptic composition comprises alcohol based compounds, non-alcohol based compounds, or a mixture of alcohol and non-alcohol based compounds.

11. A skin preparation device for applying an antiseptic composition to a skin surface of a patient, the skin preparation device comprising:
an applicator configured to absorb the antiseptic composition, the applicator comprising an application surface and a holding surface, the application surface configured to apply the antiseptic composition to the skin surface of the patient;
a holding member configured to provide a portion of the skin preparation device that is gripped by a user when using the skin preparation device, the holding member including a first end and an opposing second end, the holding member being operatively connected to the holding surface of the applicator; and
a packaging web operatively connected to the holding member to seal the applicator between the packaging web and the holding member.

12. The skin preparation device of claim 11, wherein the holding member is made of a material that provides flexibility to allow the first end and the second end to be pulled into contact with one another to be gripped by the user of the skin preparation device.

13. The skin preparation device of claim 11, wherein the packaging web comprises a peel tab to remove the packaging web from the holding member.

14. The skin preparation device of claim 11, wherein the packaging web comprises at least one perforation line to assist in removing at least a portion of the packaging web from the skin preparation device.

15. The skin preparation device of claim 11, wherein the packaging web is heat sealed to a perimeter of the holding member.

16. The skin preparation device of claim 11, wherein the packaging web is made of blister packaging.

17. The skin preparation device of claim 11, wherein the applicator is made of foam or non-woven paper.

18. The skin preparation device of claim 11, wherein the applicator has a thickness that prevents the holding member from contacting the skin surface of the patient.

19. The skin preparation device of claim 11, wherein the applicator includes a flexibility to bend as the first end and the second end of the holding member are pulled into contact with one another.

20. The skin preparation device of claim 19, wherein the holding surface of the applicator defines a plurality of grooves and ridges to assist the applicator in bending.
